(19) United States
(12) Patent Application Publication
(10) Pub. No.: US 2023/0011984 A1
(43) Pub. Date: Jan. 12, 2023

(54) PORTABLE CULTURE TEST APPARATUS

(71) Applicant: Ming Li, Changsha (CN)

(72) Inventors: MING LI, CHANGSHA (CN); HONGDUO ZHANG, CHANGSHA (CN); YONGWEI XU, SHENZHEN (CN); SIHUI DAI, CHANGSHA (CN); ZHONGRUI ZHOU, CHANGSHA (CN)

(21) Appl. No.: 17/857,575

(22) Filed: Jul. 5, 2022

(30) Foreign Application Priority Data
Jul. 6, 2021 (JP) 2021-112155

(51) Int. Cl. C12M 3/00; G01N 21/33; G01N 33/483; C12M 1/34

(52) U.S. Cl. CPC C12M 23/54; G01N 21/33; G01N 33/4833; C12M 41/12; G01N 2201/0221; G01N 2201/0231

(57) ABSTRACT

A portable culture test apparatus, comprising: a mounting portion on which a culture dish can be mounted; a vibrating portion that vibrates the mounting portion; a temperature adjusting mechanism that adjusts the temperature of the mounting portion; and an ultraviolet lamp that irradiates the culture dish mounted on the mounting portion with ultraviolet.

### FIELD OF THE DISCLOSURE

The present invention relates to a portable culture test apparatus.

### BACKGROUND OF THE DISCLOSURE

In order to promote the growth of fish germ cells, it is necessary to immediately perform deactivation treatment and distribution homogenization treatment on the germ cells which have been collected in a predetermined low temperature environment. Conventionally, in order to perform these treatments outdoors, it is necessary to carry devices such as a shaker, an ultraviolet lamp, a cold insulator, and a light shielding plate.

### SUMMARY OF THE DISCLOSURE

However, it is inconvenient to carry these plural devices, and it is difficult to simultaneously perform deactivation treatment and distribution homogenization treatment of germ cells. In addition, the accuracy of temperature adjustment by cold insulator is low.

The present invention has been made in view of such problems, and it is the object of the present invention to provide a portable culture test apparatus capable of adjusting temperature with high accuracy, performing deactivation treatment of germ cells, and homogenizing a germ cell distribution.

According to one aspect of the present invention, a portable culture test apparatus is provided which comprises a mounting portion on which a culture dish can be mounted, a vibrating portion that vibrates the mounting portion, a temperature adjusting mechanism that adjusts the temperature of the mounting portion; and an ultraviolet irradiating portion that irradiates the culture dish mounted on the mounting portion with ultraviolet.

According to this aspect, it is possible to adjust temperature with high accuracy, to perform deactivation treatment of germ cells, and to homogenize a germ cell distribution.

### BRIEF DESCRIPTION OF THE DRAWINGS

FIG. 1 is a schematic configuration diagram illustrating a portable culture test apparatus according to this embodiment.

FIG. 2 is a perspective view illustrating a principal section of an elevating mechanism.

FIG. 3 is a bottom view of a heat transfer plate.

FIG. 4 is a schematic configuration diagram of a switch portion.

FIG. 5 is a flowchart showing each step of a germ cell culture test.

### DETAILED DESCRIPTION OF THE EXEMPLARY EMBODIMENTS

Embodiments of the present invention (hereinafter referred to as this embodiment) will be described below with reference to the accompanying drawings.

(Portable Culture Test Apparatus)

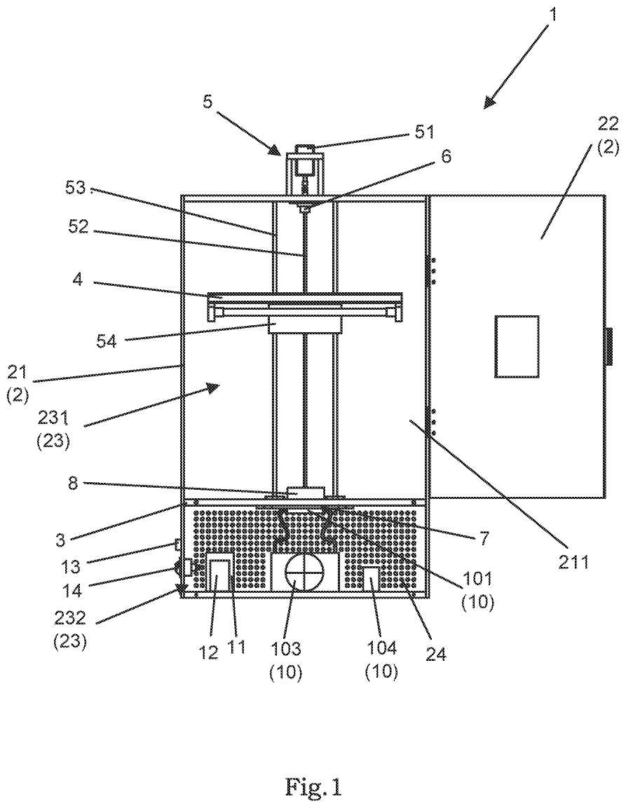

First, a portable culture test apparatus 1 according to this embodiment will be described in detail with reference to FIGS. 1 to 4.

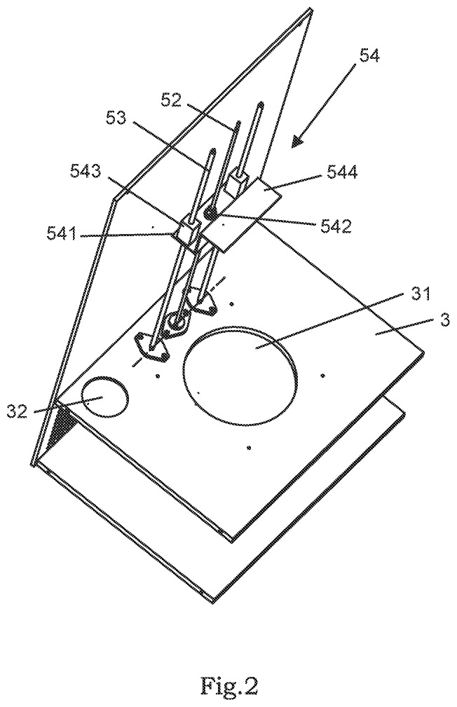

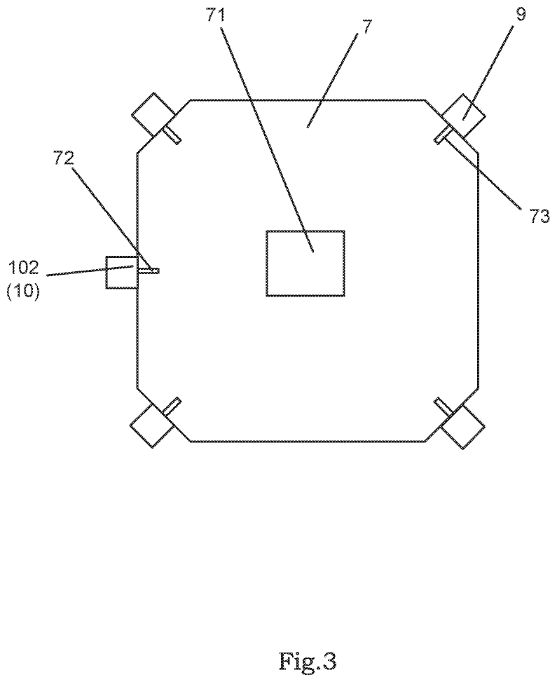

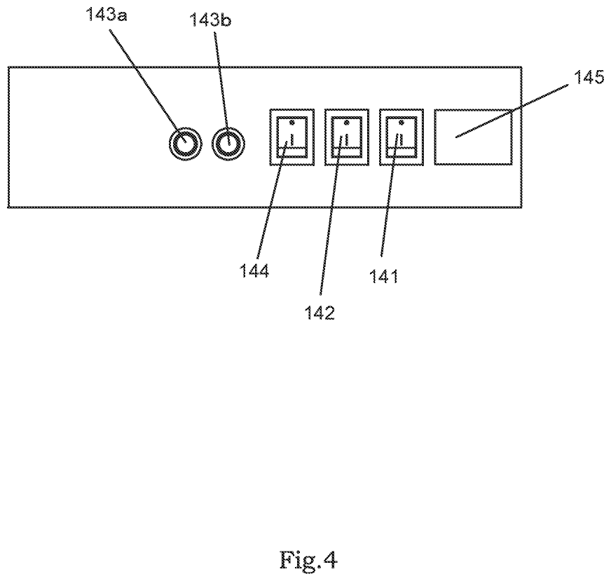

FIG. 1 is a schematic configuration diagram illustrating the portable culture test apparatus 1. FIG. 2 is a perspective view illustrating a principal section of an elevating mechanism 5. FIG. 3 is a bottom view of a heat transfer plate 7. FIG. 4 is a schematic configuration diagram of a switch portion 14. In the drawing, the left-right direction (width direction), front-back direction (depth direction), and up-down direction (vertical direction) of the portable culture test apparatus 1 are set to be directions along the X-axis, the Y-axis, and the Z-axis, respectively. In the following, for convenience of explanation, the left-right direction (width direction), front-back direction (depth direction) and up-down direction (vertical direction) of the portable culture test apparatus 1 are simply referred to as the left-right direction (width direction), front-back direction (depth direction) and up-down direction (vertical direction).

The portable culture test apparatus 1 is an apparatus for performing a culture test of germ cells (specifically, fish germ cells). As illustrated in FIG. 1, the portable culture test apparatus 1 includes a case 2, a partition plate 3, an ultraviolet lamp 4 as an ultraviolet irradiating portion, an elevating mechanism 5, a camera 6 as an imaging portion, the heat transfer plate 7 as a mounting portion, a culture dish 8, vibrating portions 9 (see FIG. 3), a temperature adjusting mechanism 10, a battery 11 as a power supply portion, a relay 12, a temperature display portion 13, and a switch portion 14 as an operation portion.

The case 2 is an accommodating portion for accommodating each component. The case 2 includes a front open box type case body 21 in which an opening 211 is formed on the front surface, and a door 22 which is hinged to the case body 21 and can be opened and closed with respect to the opening 211.

From the viewpoint of preventing ultraviolet from being irradiated to a human body from the ultraviolet lamp 4, it is preferable to configure the case body 21 and the door 22 of a material or color (for example, black) capable of blocking ultraviolet. Further, from the viewpoint of both preventing ultraviolet irradiation and reducing the weight of the entire portable culture test apparatus 1, it is preferable to configure the case body 21 and the door 22 of black acrylic.

The space surrounded by the case body 21 and the door 22 that seals the opening 211 of the case body 21 is an accommodating space 23 for accommodating each component. The accommodating space 23 is divided into a work chamber 231 and a control chamber 232 by the horizontally arranged partition plate 3. Further, the opening 211 is not formed on the control chamber 232, and is formed only on the front surface of the work chamber 231.

The work chamber 231 is a space located above the partition plate 3 and accommodates the ultraviolet lamp 4, a part of the elevating mechanism 5, and the camera 6. The control chamber 232 is a space located below the partition plate 3 and accommodates the heat transfer plate 7, the vibrating portions 9, the temperature adjusting mechanism 10, the battery 11 and the relay 12. Thus, it is possible to prevent the raising and lowering of the ultraviolet lamp 4 by the elevating mechanism 5 from being hindered by the layout of the heat transfer plate 7, the vibrating portions 9, and the temperature adjusting mechanism 10.

A plurality of heat dissipating holes 24 are formed on at least one of the front plate and the back plate of the case body 21 which is an outer wall of the control chamber 232 (see FIG. 1). Thus, since ultraviolet from the ultraviolet lamp 4 accommodated in the work chamber 231 is blocked by the partition plate 3 for partitioning the work chamber 231 and the control chamber 232, it is possible to prevent ultraviolet from entering the control chamber 232. As a result, even when the heat dissipating holes 24 are formed on the outer wall of the control chamber 232, it is possible to prevent ultraviolet from leaking to the outside through the heat dissipating holes 24.

Further, the dimension of the work chamber 231 in the up-down direction is larger than the dimension of the control chamber 232 in the up-down direction. Thus, the movable region of the elevating mechanism 5 can be increased without increasing the overall dimension of the case body 21 in the up-down direction.

As illustrated in FIG. 2, a circular through hole 31 through which the culture dish 8 can penetrate is formed at the center of the partition plate 3. Further, an opening 32 is formed at a corner of the partition plate 3, and cables (not illustrated) for electrically connecting the components accommodated in the work chamber 231 (for example, the ultraviolet lamp 4, the elevating mechanism 5 and the camera 6, etc.) and the components accommodated in the control chamber 232 (for example, the relay 12 and the switch portion 14, etc.) can penetrate the opening 32.

The ultraviolet lamp 4 irradiates the culture dish 8 mounted on the heat transfer plate 7 located below with ultraviolet. Thereby, a deactivation treatment of the germ cells in the culture dish 8 is performed.

The elevating mechanism 5 raises and lowers the ultraviolet lamp 4. Thereby, the irradiation intensity of ultraviolet on the culture dish 8 can be adjusted. Specifically, the elevating mechanism 5 can separate the ultraviolet lamp 4 from the culture dish 8 and weaken the ultraviolet irradiation to the culture dish 8 by raising the ultraviolet lamp 4. On the other hand, the elevating mechanism 5 can bring the ultraviolet lamp 4 close to the culture dish 8 and strengthen the ultraviolet irradiation to the culture dish 8 by lowering the ultraviolet lamp 4.

As illustrated in FIGS. 1 and 2, the elevating mechanism 5 includes a motor 51 as a driving portion, a lead screw 52, a pair of guide poles 53, and a carriage 54.

The motor 51 drives the lead screw 52. The motor 51 is provided on the upper surface of the top plate of the case body 21. Thus, the degree of freedom in arranging the camera 6 which is provided on the lower surface of the top plate of the case body 21 can be improved as compared with the case where the motor 51 is provided on the lower surface of the top plate of the case body 21, and it becomes easier to release the heat generated during the operation of the motor 51 to the outside. Further, since the motor 51 is provided on the top plate of the case body 21, it is not necessary to provide the motor 51 in the control chamber 232, and the degree of freedom in arranging the heat transfer plate 7, the vibrating portions 9, the temperature adjusting mechanism 10, the battery 11, and the relay 12, which are accommodated in the control chamber 232, can be improved.

The lead screw 52 is provided in a manner of extending along the vertical direction. Further, one end (upper end) of the lead screw 52 is connected to the drive shaft of the motor 51, and the other end (lower end) of the lead screw 52 is supported by the partition plate 3. Thus, since the other end of the lead screw 52 is supported by the partition plate 3, the length of the lead screw 52 can be shortened while increasing the elevating region of the ultraviolet lamp 4.

The pair of guide poles 53 guides the carriage 54 to move up and down. The pair of guide poles 53 is provided between the top plate of the case body 21 and the partition plate 3 in a manner of extending along the vertical direction. In this embodiment, the pair of guide poles 53 has one end (upper end) supported by the top plate of the case body 21 and the other end (lower end) supported by the partition plate 3, but the present invention is not limited to this, and for example, the pair of guide poles 53 may be supported by the back plate of the case body 21 via a support member or the like.

The other end (lower end) of the lead screw 52 and the other end (lower end) of the pair of guide poles 53 are located between the through hole 31 and the back plate of the case body 21 (see FIG. 2).

The carriage 54 includes a carriage body 541, a screw nut 542, a pair of slides 543 and a lamp support plate 544. The screw nut 542, the pair of slides 543 and the lamp support plate 544 are connected to the carriage body 541.

The screw nut 542 is screwed on the lead screw 52. The pair of slides 543 fits the pair of guide poles 53 and is guided by the pair of guide poles 53, respectively. The ultraviolet lamp 4 is integrally configured with the carriage 54 using the lamp support plate 544. Thus, the carriage 54 screwed on the lead screw 52 can be raised and lowered together with the ultraviolet lamp 4 as the motor 51 drives the lead screw 52.

The camera 6 images the culture dish 8 mounted on the heat transfer plate 7. The camera 6 is provided above the heat transfer plate 7 with a lens 61 facing downward. In addition, the camera 6 is connected to a terminal (not illustrated) such as a notebook PC, and the image of the inside of the imaged culture dish 8 can be displayed on the terminal. Thus, the condition of germ cells in the culture dish 8 can be observed in real time using the camera 6 and the terminal.

Specifically, the camera 6 is provided on the top plate of the case body 21. Further, the through hole 31 of the partition plate 3 is formed directly under the camera 6. Thus, the camera 6 can easily image the culture dish 8 mounted on the heat transfer plate 7 through the through hole 31.

Further, the camera 6 is provided in a manner of not interfering with the ultraviolet lamp 4, the lead screw 52, the guide poles 53 and the carriage 54 in a plan view. The camera 6 can image the culture dish 8 without being disturbed by the lead screw 52, the ultraviolet lamp 4 and the carriage 54.

The heat transfer plate 7 is for mounting the culture dish 8. The heat transfer plate 7 is provided below the partition plate 3 in a manner of corresponding to the position of the through hole 31 of the partition plate 3. That is, the heat transfer plate 7 is provided in a manner that the culture dish 8 is mounted through the through hole 31. Further, from the viewpoint of achieving good heat conduction, the heat transfer plate 7 is preferably made of a material such as aluminum. The area of the upper surface of the heat transfer plate 7 is smaller than the area of the upper surface of the partition plate 3. In this embodiment, the heat transfer plate 7 is supported by the bottom plate of the case body 21 via a support member (not illustrated), but the present invention is not limited to this, and for example, the heat transfer plate 7 may be supported by the partition plate 3 via another support member or the like.

As illustrated in FIG. 3, a recess 71 for attaching a semiconductor cooling chip 101 (see FIG. 1) constituting the temperature adjusting mechanism 10 is formed at the center of the bottom surface of the heat transfer plate 7. A sensor inserting groove 72 for inserting and attaching a temperature sensor 102 constituting the temperature adjusting mechanism 10 is formed on an edge of the heat transfer plate 7. Vibrating portion inserting grooves 73 for inserting and attaching the vibrating portions 9 are formed at the four corners of the heat transfer plate 7.

The vibrating portions 9 vibrate the heat transfer plate 7. Thus, it is possible to perform a distribution homogenization treatment of the germ cells in the culture dish 8 mounted on the heat transfer plate 7.

The temperature adjusting mechanism 10 is for adjusting the temperature of the heat transfer plate 7. The temperature adjusting mechanism 10 includes the semiconductor cooling chip 101, the temperature sensor 102, a fan 103 and a temperature controlling portion 104.

The semiconductor cooling chip 101 cools the heat transfer plate 7, and is attached to the recess 71 in a manner that the cooling surface of the semiconductor cooling chip 101 is in contact with the bottom surface of the recess 71 of the heat transfer plate 7. The temperature sensor 102 detects the temperature of the heat transfer plate 7 (that is, the temperature of the culture dish 8), and is inserted and attached to the sensor inserting groove 72 of the heat transfer plate 7.

The fan 103 is for releasing the heat in the control chamber 232 to the outside through the heat dissipating holes 24. The fan 103 is provided on the bottom plate of the case body 21 in a manner that the front surface of the fan 103 faces the front plate of the case body 21 and the fan 103 is located directly under the heat transfer plate 7.

The temperature controlling portion 104 is provided on the bottom plate of the case body 21. The temperature controlling portion 104 controls the need to drive the semiconductor cooling chip 101 and the fan 103 based on the temperature of the heat transfer plate 7 (that is, the temperature of the culture dish 8) detected by the temperature sensor 102. Further, the temperature controlling portion 104 is a controller composed of a CPU.

Specifically, when the temperature of the heat transfer plate 7 (that is, the temperature of the culture dish 8) detected by the temperature sensor 102 is higher than a predetermined temperature which is set in advance, the temperature controlling portion 104 drives the semiconductor cooling chip 101 and the fan 103. On the other hand, when the temperature of the heat transfer plate 7 (that is, the temperature of the culture dish 8) detected by the temperature sensor 102 is equal to or lower than the predetermined temperature which is set in advance, the temperature controlling portion 104 does not drive the semiconductor cooling chip 101 and the fan 103. Thus, the germ cells in the culture dish 8 can always be maintained in an appropriate temperature environment.

The battery 11 is for supplying electric power to the ultraviolet lamp 4, the elevating mechanism 5, the camera 6, the temperature adjusting mechanism 10, the temperature display portion 13 and the switch portion 14 through the relay 12. The battery 11 is configured to be rechargeable. Thus, the portable culture test apparatus 1 can be operated even outdoors without an electrical outlet. In addition, the battery 11 is provided on the bottom plate of the case body 21 in a manner of being adjacent to the relay 12. Further, the fan 103 is arranged between the temperature controlling portion 104 and the relay 12.

In this embodiment, the electric power of the portable culture test apparatus 1 is supplied from the rechargeable battery 11, but the present invention is not limited to this, and for example, the electric power may be supplied from a notebook PC or an external power source, etc. In this case, the battery 11 can be omitted.

The temperature display portion 13 displays the temperature of the heat transfer plate 7 (that is, the temperature of the culture dish 8) detected by the temperature sensor 102. This allows the user to observe the temperature of the heat transfer plate 7 (that is, the temperature of the culture dish 8) displayed on the temperature display portion 13. The temperature display portion 13 is provided on an outer surface of a side plate (specifically, the left side plate) of the case body 21, that is, an outer wall of the control chamber 232.

The switch portion 14 is provided on an outer surface of a side plate (specifically, the left side plate) of the case body 21, which is an outer wall of the control chamber 232, in a manner of being adjacent to the temperature display portion 13. Specifically, as illustrated in FIG. 4, the switch portion 14 includes a power switch 141, an ultraviolet lamp switch 142, elevating position adjusting switches 143, a vibrating portion switch 144, and a temperature setting switch 145.

The power switch 141 is a switch for switching on or off the entire portable culture test apparatus 1. The ultraviolet lamp switch 142 is a switch for switching on or off the ultraviolet lamp 4. The elevating position adjusting switches 143 are switches for adjusting the elevating position of the ultraviolet lamp 4, and include a raising switch 143a and a lowering switch 143b. The vibrating portion switch 144 is a switch for switching on or off the vibrating portions 9. The temperature setting switch 145 is a switch for setting a predetermined temperature.

(Germ Cell Culture Test)

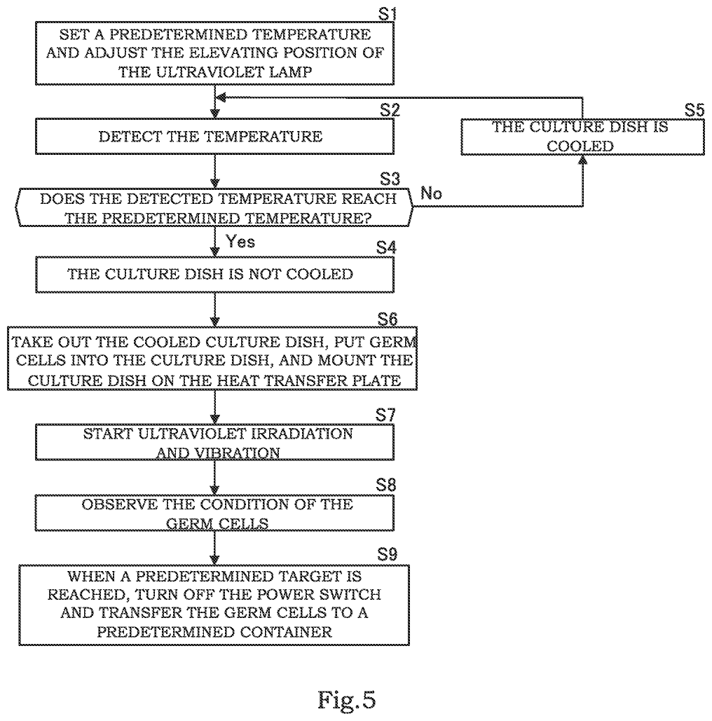

Next, a germ cell culture test using the portable culture test apparatus 1 will be described in detail with reference to FIG. 5.

FIG. 5 is a flowchart showing each step of the germ cell culture test.

Thus, when the power switch 141 is switched from off to on, the germ cell culture test using the portable culture test apparatus 1 starts.

As shown in FIG. 5, first, in Step S1, the user sets a predetermined temperature by operating the temperature setting switch 145 and adjusts the elevating position of the ultraviolet lamp 4 by operating the elevating position adjusting switches 143, and then the process proceeds to Step S2.

Next, in Step S2, the temperature sensor 102 detects the temperature of the heat transfer plate 7 (that is, the temperature of the culture dish 8) and outputs the detected temperature of the heat transfer plate 7 (that is, the temperature of the culture dish 8) to the temperature controlling portion 104, and then the process proceeds to Step S3.

Next, in Step S3, the temperature controlling portion 104 determines whether or not the temperature of the heat transfer plate 7 (that is, the temperature of the culture dish 8) detected by the temperature sensor 102 has reached the predetermined temperature. If the temperature of the heat transfer plate 7 (that is, the temperature of the culture dish 8) detected by the temperature sensor 102 has reached the predetermined temperature (if Yes), the process proceeds to Step S4. On the other hand, if the temperature of the heat transfer plate 7 (that is, the temperature of the culture dish 8) detected by the temperature sensor 102 has not reached the predetermined temperature (if No), the process proceeds to Step S5.

Next, in Step S4, the temperature controlling portion 104 executes control so as not to drive the semiconductor cooling chip 101 and the fan 103 based on the determination result of Yes in Step S3, and then the process proceeds to Step S6. Specifically, in Step S4, the temperature controlling portion 104 generates a non-drive signal based on the determination result of Yes in Step S3. Then, the temperature controlling portion 104 outputs the generated non-drive signal to the semiconductor cooling chip 101 and the fan 103. Thus, the semiconductor cooling chip 101 and the fan 103 are not driven, and the heat transfer plate 7 and the culture dish 8 are not cooled.

On the other hand, in Step S5, the temperature controlling portion 104 executes control so as to drive the semiconductor cooling chip 101 and the fan 103 based on the determination result of No in Step S3, and then the process returns to Step S2. Specifically, in Step S5, the temperature controlling portion 104 generates a drive signal based on the determination result of No in Step S3. Then, the temperature controlling portion 104 outputs the generated drive signal to the semiconductor cooling chip 101 and the fan 103. Thus, the semiconductor cooling chip 101 and the fan 103 are driven to cool the heat transfer plate 7 and the culture dish 8.

Next, in Step S6, the user takes out the culture dish 8, which is mounted on the heat transfer plate 7 and cooled by the semiconductor cooling chip 101 and the fan 103, from the portable culture test apparatus 1, puts germ cells into the culture dish 8, and mounts the culture dish 8, into which germ cells have been put, on the heat transfer plate 7, and then the process proceeds to Step S7.

Next, in Step S7, the user switches the ultraviolet lamp switch 142 and the vibrating portion switch 144 of the switch portion 14 from off to on to vibrate the culture dish 8 by the vibrating portions 9 while irradiating the culture dish 8 with ultraviolet by the ultraviolet lamp 4 and start the treatment of distribution homogenization and deactivation of the germ cells in the culture dish 8, and then the process proceeds to Step S8.

Next, in Step S8, the user performs the treatment of distribution homogenization and deactivation of the germ cells in the culture dish 8 while observing the condition of the germ cells in the culture dish 8 through the camera 6 and the terminal in a state where the camera 6 is connected to the terminal, and then the process proceeds to Step S9.

Next, in Step S9, when the user observes that the degree of distribution homogenization and the degree of deactivation of the germ cells have reached a predetermined target, the user switches the power switch 141 from on to off and transfers the germ cells in the culture dish 8 to a predetermined container. This completes the germ cell culture test.

(Actions and Effects)

Subsequently, major actions and effects of this embodiment will be described.

The portable culture test apparatus 1 according to this embodiment includes: the heat transfer plate 7 on which the culture dish 8 can be mounted; the vibrating portions 9 that vibrate the heat transfer plate 7; the temperature adjusting mechanism 10 that adjusts the temperature of the heat transfer plate 7; and the ultraviolet lamp 4 that irradiates the culture dish 8 mounted on the heat transfer plate 7 with ultraviolet.

According to this, the temperature adjusting mechanism 10 can accurately adjust the temperature of the germ cells in the culture dish 8 mounted on the heat transfer plate 7 by adjusting the temperature of the heat transfer plate 7. In addition, the vibrating portions 9 can perform the distribution homogenization treatment of the germ cells in the culture dish 8 mounted on the heat transfer plate 7 by vibrating the heat transfer plate 7. Further, the ultraviolet lamp 4 can perform the deactivation treatment of germ cells by irradiating the culture dish 8 mounted on the heat transfer plate 7 with ultraviolet. Thus, it is possible to adjust the temperature with high accuracy, to perform the deactivation treatment of germ cells, and to homogenize the germ cell distribution.

In this embodiment, the portable culture test apparatus 1 further includes the camera 6 which is provided above the heat transfer plate 7 and images the culture dish 8 mounted on the heat transfer plate 7.

According to this, it is possible to perform the deactivation treatment and the distribution homogenization treatment of germ cells while observing the condition of the germ cells in the culture dish 8.

In this embodiment, the portable culture test apparatus 1 further includes the elevating mechanism 5 that raises and lowers the ultraviolet lamp 4 in a manner that the ultraviolet lamp 4 is located above the heat transfer plate 7.

According to this, the irradiation intensity of ultraviolet on the germ cells in the culture dish 8 can be adjusted.

In this embodiment, the portable culture test apparatus 1 further includes: the case 2; and the partition plate 3 for partitioning the case 2 into the work chamber 231 and the control chamber 232 located below the work chamber 231, wherein the through hole 31 through which the culture dish 8 can penetrate is formed on the partition plate 3, and the work chamber 231 accommodates the ultraviolet lamp 4 and the elevating mechanism 5, and the control chamber 232 accommodates the heat transfer plate 7, the vibrating portions 9, and the temperature adjusting mechanism 10.

According to this, the ultraviolet lamp 4 and the elevating mechanism 5 are accommodated in the work chamber 231, and the heat transfer plate 7, the vibrating portions 9 and the temperature adjusting mechanism 10 are accommodated in the control chamber 232, and thus, it is possible to prevent the raising and lowering of the ultraviolet lamp 4 by the elevating mechanism 5 from being hindered by the layout of the heat transfer plate 7, the vibrating portions 9, and the temperature adjusting mechanism 10. Further, since ultraviolet from the ultraviolet lamp 4 accommodated in the work chamber 231 is blocked by the partition plate 3, it is possible to prevent ultraviolet from entering the control chamber 232.

In this embodiment, the elevating mechanism 5 includes the motor 51 provided above the ultraviolet lamp 4, and the temperature adjusting mechanism 10 is provided below the partition plate 3.

According to this, since the motor 51 is provided not below the partition plate 3 but on the top plate of the case 2, the degree of freedom in arranging the temperature adjusting mechanism 10, which is provided below the partition plate 3, can be improved.

In this embodiment, the elevating mechanism 5 further includes the lead screw 52 with one end connected to the motor 51 and the other end supported by the partition plate 3.

According to this, since the other end of the lead screw 52 is supported by the partition plate 3, the length of the lead screw 52 can be shortened while increasing the elevating region of the ultraviolet lamp 4.

While the embodiments of the present invention have been described above, the above-described embodiments only show part of application examples of the present invention and are not intended to limit the technical scope of the present invention to the specific configurations of the above-described embodiments.

This application claims priority based on Japanese Patent Application No. 2021-112155 filed with the Japan Patent Office on 6 Jul. 2021, the entire contents of this application are incorporated herein by reference.

1. A portable culture test apparatus, comprising:
a mounting portion on which a culture dish can be mounted;
a vibrating portion that vibrates the mounting portion;
a temperature adjusting mechanism that adjusts the temperature of the mounting portion; and
an ultraviolet irradiating portion that irradiates the culture dish mounted on the mounting portion with ultraviolet.

2. The portable culture test apparatus according to claim 1, further comprising:
an imaging portion that is provided above the mounting portion and images the culture dish mounted on the mounting portion.

3. The portable culture test apparatus according to claim 2, further comprising:
an elevating mechanism that raises and lowers the ultraviolet irradiating portion in a manner that the ultraviolet irradiating portion is located above the mounting portion.

4. The portable culture test apparatus according to claim 3, further comprising:
a case; and
a partition plate for partitioning the case into a work chamber and a control chamber located below the work chamber with a through hole through which the culture dish can penetrate formed on the partition plate,
wherein the work chamber accommodates the ultraviolet irradiating portion and the elevating mechanism; and
the control chamber accommodates the mounting portion, the vibrating portion, and the temperature adjusting mechanism.

5. The portable culture test apparatus according to claim 4, wherein:
the elevating mechanism includes a driving portion provided on a top plate of the case; and
the temperature adjusting mechanism is provided below the partition plate.

6. The portable culture test apparatus according to claim 5, wherein:
the elevating mechanism further includes a lead screw with one end connected to the driving portion and the other end supported by the partition plate.
